(19) United States
(12) Patent Application Publication
(10) Pub. No.: US 2016/0008409 A1
(43) Pub. Date: Jan. 14, 2016

(54) SUPERCRITICAL CARBON DIOXIDE TISSUE PROCESSING METHODS

(71) Applicant: AlloSource, Centennial, CO (US)

(72) Inventors: Arthur Joslyn, Centennial, CO (US); Timothy Walden, Aurora, CO (US)

(73) Assignee: AlloSource, Centennial, CO (US)

(21) Appl. No.: 14/794,354

(22) Filed: Jul. 8, 2015

Related U.S. Application Data
(60) Provisional application No. 62/022,402, filed on Jul. 9, 2014.

(51) Int. Cl. A61K 35/50; C12N 5/073

(52) U.S. Cl. CPC A61K 35/50; C12N 5/0605

(57) ABSTRACT

Embodiments of the present invention may include a method for improving handling characteristics of an amnion tissue. The method may include providing the amnion tissue from a human donor. Furthermore, the method may include exposing the amnion tissue to supercritical carbon dioxide to form an exposed amnion tissue. The exposed amnion tissue may have a higher ultimate tensile strength and elastic modulus than the amnion tissue before the exposure.

### CROSS-REFERENCE TO RELATED APPLICATION

This application claims the benefit of U.S. Provisional Application No. 62/022,402, filed Jul. 9, 2014, the entire contents of which are incorporated herein by reference for all purposes.

### BACKGROUND OF THE INVENTION

Embodiments of the present invention are directed in general to the field of medical dressings, and in particular to methods for preparing tissue compositions.

Many types of human tissue can be used to help treat a variety of ailments, including for wound care and burn care. These tissues include dermal, fascia, and birth tissue. Human birth tissue can be defined as the amniotic sac (which includes two tissue layers, the amnion and chorion), the placenta, the umbilical cord, and the cells of fluid contained in each. Human amniotic membrane has been used for many years in various surgical procedures, including skin transplantation and ocular surface disorder treatments to prevent adhesions. Lately, certain known medical techniques involve the application of tissue to patients in the form of surgical dressings. Although tissue compositions and methods are presently available and provide real benefits to patients in need thereof, many advances may still be made providing improved dressing systems and manufacturing methods. The dressing systems and manufacturing methods described herein provide further solutions and answers to these outstanding needs.

### BRIEF SUMMARY OF THE INVENTION

Tissue dressings, such as amniotic tissue dressings, can be used to treat patients. Such dressings can be used to treat patients having tarsal tunnel syndrome, iliotibial band stenosis, phantom pain associated with amputation, damaged meniscus, peripheral nerve damage or injuries, and the like. Further, dressings can be used in spinal treatments including laminectomies, anterior lumbar interbody fusion (ALIF) procedures, laminotomies, and in extensor halgus longus tendon surgeries.

These tissue dressings may be used as adhesion barriers. Adhesiogenesis may occur after surgical repair of orthopedic, neurological, gynecological, gastrointestinal, and other surgeries. When a tissue is disrupted during surgical repair, biomolecules may migrate to the surgical site and cause adhesions to develop, which may cause pain and discomfort. Tissue dressing may prevent surgical adhesions and thus may prevent the need for follow-up surgeries to lyse or otherwise remove adhesions. These tissue dressings may be wrapped around a tendon, nerve, or other structure to prevent or mitigate post-surgical adhesion formation.

Embodiments of this technology may produce tissue dressings that are more durable and have higher tensile strength than conventional tissue dressings. These tissue dressings may be more easily applied to a patient. Embodiments of the technology described herein encompass techniques for treating the tissue and improving its handling characteristics and providing for a stronger graft, while preserving other properties of the tissue without prohibitively significant investments in capital or time. These treatment methods may avoid a tissue that is difficult for a surgical practitioner to handle and is easily torn, crumpled, bunched up, and/or damaged before, during, and/or after administration to a patient.

In one aspect, embodiments of the present technology may include a method for improving handling characteristics of a soft tissue. The method may include providing the soft tissue from a human donor. The tissue may include amnion, fascia, or dermal tissue. In embodiments, the tissue may be amnion tissue. In some cases, amnion tissue may be provided in any of a variety of constructs, including amnion tissue configurations such as those described in U.S. Ser. No. 12/428,836 filed Apr. 23, 2009, U.S. Ser. No. 13/186,661 filed Jul. 20, 2011, U.S. Ser. No. 13/894,637 filed May 15, 2013, and U.S. Ser. No. 13/793,331 filed Mar. 11, 2013, the entire contents of each of which are incorporated herein by reference.

The method may also include exposing the amnion tissue to a supercritical carbon dioxide (SCCO₂) to form an exposed amnion tissue. Exposing the amnion tissue to SCCO₂ may be at a temperature from 32° C. to 38° C. The exposure temperature may be about 35° C. The range of pressures for the exposure of the amnion tissue to SCCO₂ may be from 1,350 to 1,475 psi. The exposure time may be from 1.5 to 4.5 hours, including, for example, about 3 hours.

The method may further include exposing the amnion tissue to additives during the exposure of the amnion tissue to SCCO₂. These additives may include peracetic acid, hydrogen peroxide, or ethanol. The volume of the additives may be greater than or equal to 16 mL. The method may further include sterilizing the amnion tissue without supercritical carbon dioxide.

The exposed amnion tissue may have a higher tensile strength than the amnion tissue before exposure. The amnion tissue may be less likely to tear than amnion tissue that had not been treated with SCCO₂. The exposed soft tissue may have an ultimate tensile strength greater than 20 kPa, 25 kPa, 30 kPa, 35 kPa, or 40 kPa in embodiments. In some instances, the UTS for treated tissue may increase over untreated tissue by greater than 40%, 50%, 60%, 70%, or 80%. The exposed soft tissue may have an elastic modulus greater than 90 kPa, 100 kPa, 110 kPa, or 120 kPa in embodiments. In some instances, the EM for treated tissue may increase over untreated tissue by greater than 40%, 50%, 60%, 70%, or 80%.

In another aspect, embodiments of the present technology may include a method for preventing adhesions in a patient after surgery. The method may include providing an amnion tissue from a human donor. The method may also include exposing the amnion tissue to a supercritical carbon dioxide to form an exposed amnion tissue. Exposing the amnion tissue to SCCO₂ may be according to any of the methods described herein. The method may further include administering the exposed amnion tissue to the patient.

In another aspect, embodiments of this technology may include an amnion tissue composition. The amnion tissue may have been treated with a supercritical carbon dioxide. The amnion tissue may include additives, such as peracetic acid, hydrogen peroxide, or ethanol. The amnion tissue may have a different structure than amnion tissue that had not been treated with SCCO₂. The treated amnion tissue may have improved handling characteristics, including higher tensile strength or increased durability.

### BRIEF DESCRIPTION OF THE DRAWINGS

FIG. 1 shows a cross-sectional view of the tissue structure of a segment of the fetal sac according to embodiments of the present invention.

FIG. 2 shows the steps in the process of improving handling characteristics of a soft tissue according to embodiments of the present invention.

FIG. 3 shows the steps in the process of administering an amnion tissue according to embodiments of the present invention.

FIG. 4 shows a stress test on a tissue sample treated with supercritical carbon dioxide according to embodiments of the present invention.

FIG. 5 shows a stress test on a tissue sample not treated with supercritical carbon dioxide according to embodiments of the present invention.

### DETAILED DESCRIPTION OF THE INVENTION

Embodiments of the present invention encompass methods for processing tissue for use in wound coverings, burn care barrier membranes, anti-adhesion, tendon repair, rotator cuff repair, hernia repair, and other potential uses. Methods may provide a soft tissue with better strength and handling properties than available with other techniques. Soft tissues produced by these methods may not be thin, may have high tensile strength, and may not result in self-adhesion. In addition, such treatment methods may allow for amnion tissue to have desirable properties such as pliability, suppleness, and clinginess when rehydrated. These tissues may easily be applied to and adhere to wounds or other treatment areas. Embodiments include using SCCO₂ to alter or enhance, instead of merely maintaining, the mechanical and handling properties of amniotic or other soft tissue allografts.

The values for elastic modulus (EM) and ultimate tensile strength (UTS) for certain materials are shown in Table 1. The values for bone, pine wood, nylon-6, polypropylene, and rubber are from Tensile Modulus—Modulus of Elasticity or Young's Modulus—for some common materials, The Engineering Toolbox, http://www.engineeringtoolbox.com/young-modulus-d_{—}417.html, incorporated herein by reference. The values for human skin are from A. J. Gallagher et al., “Dynamic Tensile Properties of Human Skin,” IRCOBI Conference 2012 (available at http://www.ircobi.org/downloads/irc12/pdf_files/59.pdf), incorporated herein by reference. As shown in Table 1, the mechanical properties vary widely across different materials. Embodiments described herein may provide advantageous mechanical properties to tissues for surgical applications.

TABLE 1
Mechanical properties of materials

| | | Ultimate Tensile Strength |
|---|---|---|
| Material | Elastic Modulus (GPa) | (MPa) |
| Bone | 18 | 170 (Compressive) |
| Pine Wood | 90 | 40 |
| Nylon-6 | 2-4 | 45-90 |
| Polypropylene | 1.5-2 | 28-36 |
| Rubber, Small Strain | 0.01-0.1 | |
| Human Skin | 0.99 | 0.27 |

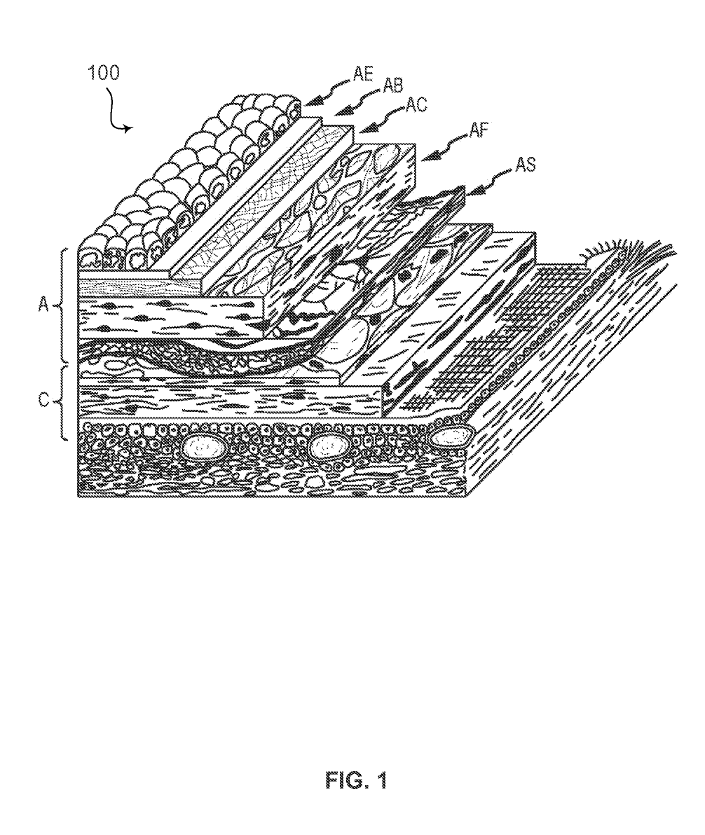

Turning to the drawings, FIG. 1 illustrates tissue features of a human fetal sac structure 100, including the anatomy of the amnion A and chorion C. As shown here, the amnion layer has several cell layers and has two sides with different cellular components. According to this depiction, the amnion A includes a single layer of ectodermally derived columnar epithelial cells AE adhered to a basement membrane AB. In turn the basement membralle AB includes collagen I, collagen III, collagen IV, laminin, glycosaminoglycans, and fibronectin, and is attached to an underlying layer of connective tissue. The connective tissue includes an acellular compact layer AC of reticular fibers, a fibroblast layer AF, and a spongy layer AS (referred to as Wharton's jelly) which form a network of fine fibrils surrounded by mucus. When the amnion A is separated from the chorion C, a two sided, asymmetrical tissue is produced having an epithelial layer AE with epithelial cells on one side and a fibroblast layer AF on the opposite side. Hence, the separated amnion A includes an epithelial layer AE on one side and a fibroblast layer AF on the opposing side. Between the epithelial and fibroblast layers is a basement membrane AB and a compact layer AC. The fibroblast layer may be considered to include a loose network of reticulum containing fibroblasts. The fibroblast layer also typically includes collagen (e.g. Types I, III, and VI) and glycoproteins (e.g. nidogen, laminin, and fibronectin).

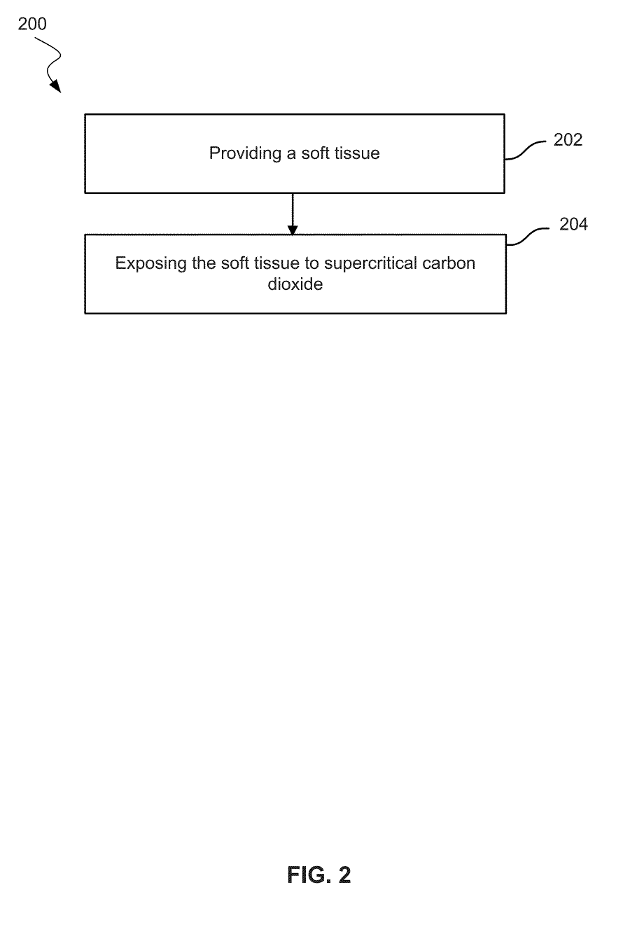

FIG. 2 shows steps in a method 200 for improving handling characteristics of a soft tissue. A soft tissue is provided in step 202. Then in step 204, the soft tissue is exposed to supercritical carbon dioxide.

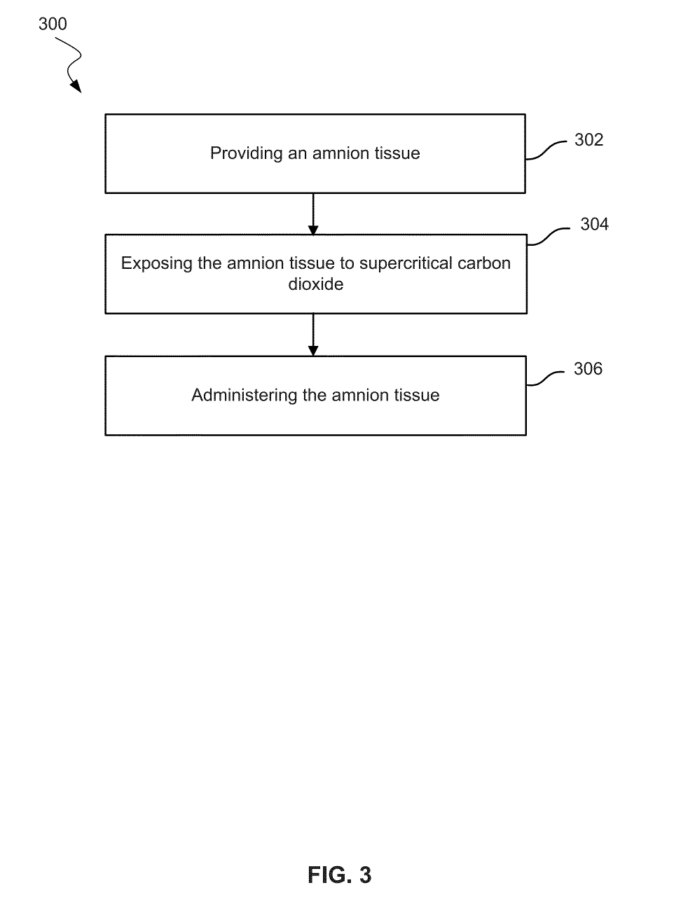

FIG. 3 shows steps in a method 300 for preventing adhesions after surgery. Step 302 includes providing an amnion tissue. Later in step 304, the amnion tissue is exposed to supercritical carbon dioxide. The amnion tissue is administered in step 306.

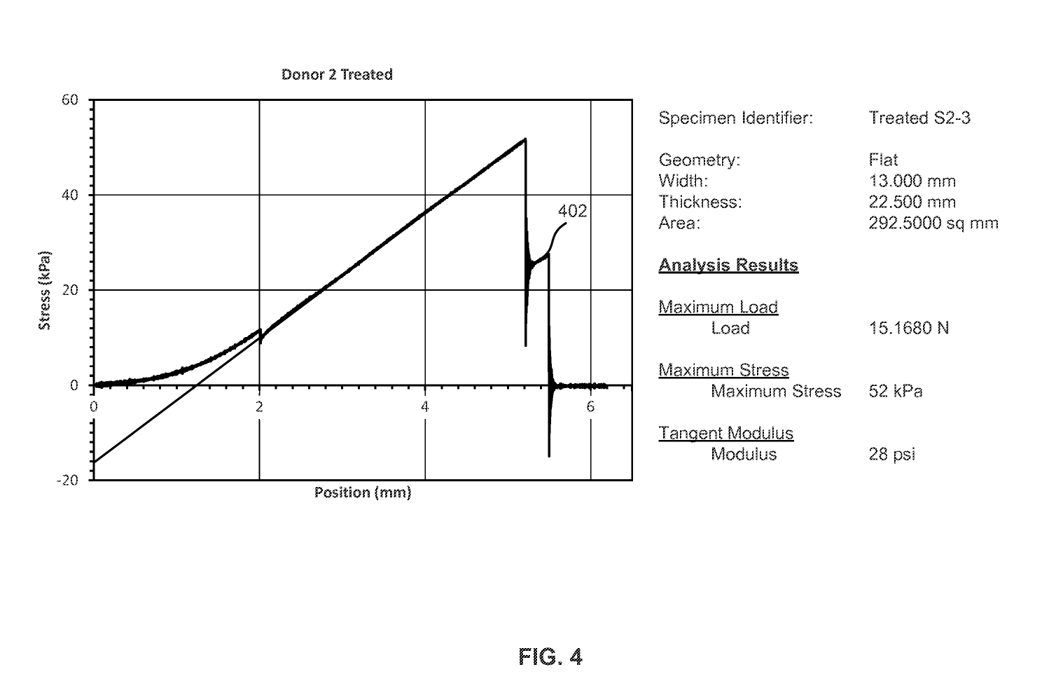

FIG. 4 shows the stress of a sample of amniotic tissue treated with supercritical carbon dioxide (SCCO₂) measured versus displacement. With no displacement, the sample has no stress. As the displacement increases, the stress increases until finally the sample breaks and the stress measures zero again. The treated sample experiences an initial drop in stress followed by an secondary rise 402 in stress before the stress drops again to zero. This phenomenon is described in greater detail in Example 2.

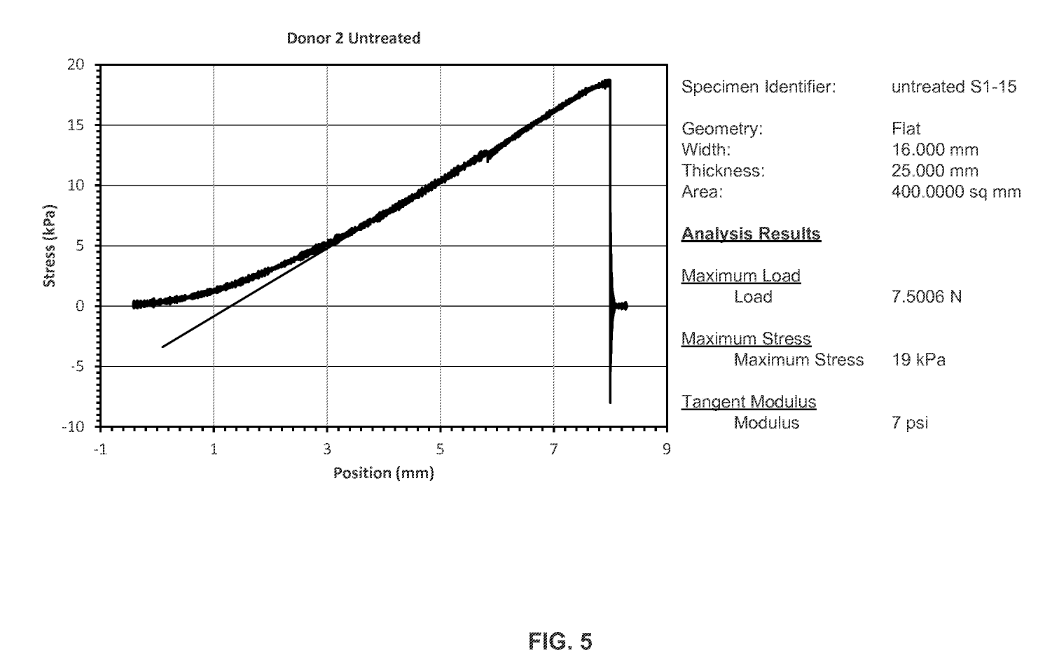

FIG. 5 shows the stress of a sample of amniotic tissue not treated with supercritical carbon dioxide measured versus displacement.

In one aspect, embodiments of this technology may include a method for improving handling characteristics of a soft tissue. The method may include providing the soft tissue from a human donor. The tissue may include amnion, fascia, dermal, and/or other soft tissues. The tissue, which may include amnion tissue and fascia tissue, may differ from some tissues used for transplant as the tissue may not be applied to or be used to treat the same type of tissue in a patient. For example, amnion tissue from a donor in embodiments of the present technology may not be used to treat amnion tissue in a patient. With returning reference to FIG. 1, the soft tissue may include amnion A separated from chorion C. Amnion tissue may be processed as AlloWrap DS™ up to its final packaging in Tyvek™. Such processing may be according to methods disclosed in U.S. application Ser. No. 13/793,331, which is incorporated herein by reference.

The method may also include exposing the soft tissue to a supercritical carbon dioxide to form an exposed soft tissue. Exposing the soft tissue to SCCO₂ may be at a temperature from 32° C. to 38° C., such as 35° C. The pressure may be from 1,350 to 1,475 psi. The temperature and pressure may be any temperature or pressure where the carbon dioxide is in a supercritical state. The exposure time may be from about 1.5 to about 4.5 hours, from about 1.5 to about 2.0 hours, from about 2.0 to about 3.0 hours, from about 3.0 to about 4.0 hours, or from about 4.0 to about 4.5 hours according to embodiments. For example, the exposure time may be about 3 hours. This exposure time may be substantially longer than the exposure times needed for sterilization of the soft tissue (e.g., the exposure time may be up to six times longer than exposure times for sterilization). The range of impeller speeds may be from 600 to 800 RPM, such as from 660 to 710 RPM.

Exposing the supercritical carbon dioxide, as described herein, may alter the characteristics of the soft tissue, while conventional use of supercritical carbon dioxide to sterilize certain tissues involves preserving the native characteristics of the tissues as much as possible. Without intending to be bound by any particular theory, it is believed that the SCCO₂ in embodiments described herein collapses the three-dimensional structure of the amnion helical protein by removing some intramolecular water. This dehydration may result in a drier and stronger tissue with improved handling characteristics. Tissues dehydrated by methods other than with SCCO₂ may not result in improved handling characteristics, including increased UTS and EM.

The method may further include exposing the soft tissue to additives during the exposure of the soft tissue to SCCO₂. These additives may include peracetic acid, hydrogen peroxide, ethanol, or other compounds. The additives may exclude any one of or any group of the compounds listed. The volume of the additives may be greater than or equal to 16 mL. The additive volume may be greater than or equal to 0.001% of the total volume of CO₂. The method may also include exposing the soft tissue to humidity. The method may further include a sterilization step that does not include SCCO₂. Alternatively, the method may not include an additional sterilization step, such as irradiation, as the treatment with SCCO₂ may provide SAL6 terminal sterilization levels. The Sterility Assurance Level (SAL) gives the probability that a given treatment sample would be non-sterile. SAL6 indicates that one unit in a million would be non-sterile and is the industry accepted definition of sterile.

The exposed soft tissue may have a higher tensile strength than the soft tissue before exposure. The soft tissue may be less likely to tear than soft tissue that had not been treated with SCCO₂. The soft tissue may have a thickness greater than 15 mm, 20 mm, 25 mm, or 30 mm in embodiments. The soft tissue may have a thickness less than 20 mm, 25 mm, 30 mm, 35 mm, or 40 mm in embodiments.

In another aspect, an embodiment of the technology may include a method for preventing adhesions in a patient after surgery. Embodiments may also include use in hernia repair, uro-gynecological slings (e.g., bladder), and other soft tissue applications. The method may include providing an amnion tissue from a human donor. The patient may include exposing the amnion tissue to a supercritical carbon dioxide to form an exposed amnion tissue, which may be performed according to any of the methods described herein. The method may further include administering the exposed amnion tissue to the patient. The patient may be different from the human donor.

In another aspect, embodiments of this technology may include an amnion tissue composition. The amnion tissue may have been treated with a supercritical carbon dioxide. The treatment may include exposure to SCCO₂ according to any of the methods described herein. The amnion tissue may include additives, such as peracetic acid, hydrogen peroxide, or ethanol.

The treated amnion tissue may have a different structure than amnion tissue that had not been treated with SCCO₂. The treated amnion tissue may have improved handling characteristics, including higher tensile strength or increased durability. Treated fascia tissue may also have similar improvements in handling characteristics.

### EXAMPLE 1

Each of the four liquid CO₂ tanks and the power strip powering a Nova 2200™ sterilizer (Novasterilis, Lansing, N.Y.) sterilizer are turned on and the vessel pressure is at zero. The vessel was opened by unclamping the split rings, moving them apart away from the vessel, and lifting up the vessel head.

The top two baskets from the vessel and the additive pad sleeve were removed. The retaining ring still elevated the bottom basket above the vessel impeller. An additive pad was inserted between the two baskets. A volume of 16 mL of chemical additive was then added to the additive pad.

The additive pad sleeve with the additive-soaked pad was placed into the small bottom basket, so that part of the pad sits over the inlet valve of the vessel. The amnion tissue samples were processed as AlloWrap DS™ and packaged in Tyvek™ packaging. Tyvek™ packaging has a paper-like side made of high density polyethylene and permeable to vapor, and Tyvek™ packaging has a plastic side that is clear and not permeable to vapor. Vapor permeability may allow the SCCO₂ to access the enclosed tissue. The tissue samples were arranged in a basket so that the sides of adjacent samples touched paper-like side to paper-like side and plastic side to plastic side. The basket was either a 7″ deep center basket or a 4″ deep top basket, depending on the size and number of tissue samples.

After the baskets were loaded, they were placed back into the vessel. The 7″ basket was placed on top of the small bottom basket, and the 4″ basket was placed on top of the 7″ basket.

A volume of 25 mL of sterile water was added to the vessel with a spray bottle once the baskets were in place. In SCCO₂ processing, sterile water may aid in inactivating spores. The vessel head was then slowly lowered to meet with the top of the vessel and set onto the vessel mating surface. The vessel head was pressed closed by hand, and the two split rings clamped the vessel and vessel head together.

The sterilizer was then set to run with an hour of humidity. The sterilizer then pumped carbon dioxide into the vessel until a temperature of approximately 35° C. and a pressure of about 1436 psi. The carbon dioxide was run for 3 hours. The range of speed of the impeller during the run was from 662 to 702 RPM.

After the run finished, the sterilizer went through a controlled depressurization of the vessel. After depressurization, the vessel was opened and the samples were removed.

### EXAMPLE 2

Tissue samples produced by Example 1 were characterized by ultimate tensile strength (UTS) testing and elastic modulus (EM) testing. Tissues that were treated with SCCO₂ from two donors were compared against tissues that were not treated with SCCO₂ from the same two donors. Sections of tissues were cut and a tension test was performed using an ADMET™ eXpert 2600 uniaxial mechanical strength tester with a 10 lb load cell.

A result from one sample test on a treated tissue from a donor is shown in FIG. 4, while a result from one sample test on an untreated tissue from the same donor is shown in FIG. 5. FIGS. 4 and 5 show the ultimate tensile strength, the highest stress that the sample can tolerate before the sample breaks and the stress measurement returns to zero. The EM is calculated by ADMET® software using a least squares fit and the dimensions of the piece of tissue. The treated sample in FIG. 4 reaches a higher maximum stress (i.e., the UTS) of 52 kPa than the untreated sample in FIG. 5 (19 kPa). The EM of the treated sample is denoted by the tangent modulus of 28 psi in FIG. 4, while the EM of the untreated sample in FIG. 5 is 7 psi.

Also shown in FIG. 4 is an initial drop in stress followed by a secondary rise in stress 402. The initial drop in stress is believed to be a result of a break in one sheet of the amniotic tissue, for AlloWrap DS™ is a two amniotic layer graft. In almost all of the treated samples, the two layers broke at separate times.

The results for several samples are shown in Table 2. The results show that the treated samples have a higher UTS and EM than the untreated samples.

TABLE 2
Results of uniaxial testing
for ultimate tensile strength and elastic modulus.

| | Donor 1 | | Donor 2 | |
|---|---|---|---|---|
| | Untreated | Treated | Untreated | Treated |
| Sample Size | 22 | 21 | 24 | 23 |
| UTS Average (kPa) | 17.95 | 25.57 | 18.50 | 31 |
| UTS Standard Deviation (kPa) | 6.48 | 8.47 | 5.04 | 12.21 |
| EM Average (kPa) | 63.58 | 103.35 | 58.85 | 108.74 |
| EM Standard Deviation (kPa) | 19.37 | 27.97 | 16.03 | 38.92 |

Further statistical analysis on the experimental results shows that the increases in UTS and EM resulting from treating with SCCO₂ are statistically significant. The samples were further analyzed using a two-sample student's t-test with unequal variance. The p values comparing different groups are show in Table 3. The statistical tests also show that there is no statistically significant difference between the two donors when comparing the treated and untreated tissues of each donor.

TABLE 3
P values for comparison of groups

| UTS donor 1 untreated to treated | 2.65 × 10⁻³ |
|---|---|
| UTS donor 2 untreated to treated | 6.12 × 10⁻³ |
| Modulus donor 1 untreated to treated | 7.10 × 10⁻⁶ |
| Modulus donor 2 untreated to treated | 5.12 × 10⁻⁶ |
| UTS untreated donor 1 to donor 2 | 0.61 |
| UTS treated donor 1 to donor 2 | 0.09 |
| Modulus untreated donor 1 to donor 2 | 0.386 |
| Modulus treated donor 1 to donor 2 | 0.606 |

A higher UTS may result in increased strength for holding together a surgical repair. A higher EM may result in a slightly stiffer material, which may be easier to place in a wound or surgical site without wrinkles and/or other unwanted folding. The higher EM and UTS does not result in a material that is too stiff for surgical applications. This example demonstrates that treated samples possess superior characteristics for surgical applications.

Having described several embodiments, it will be recognized by those of skill in the art that various modifications, alternative constructions, and equivalents may be used without departing from the spirit of the invention. Additionally, a number of well-known processes and elements have not been described in order to avoid unnecessarily obscuring the present invention. Additionally, details of any specific embodiment may not always be present in variations of that embodiment or may be added to other embodiments.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a method” includes a plurality of such methods and reference to “the tissue” includes reference to one or more tissues and equivalents thereof known to those skilled in the art, and so forth. The invention has now been described in detail for the purposes of clarity and understanding. However, it will be appreciated that certain changes and modifications may be practice within the scope of the appended claims.

1. A method for improving handling characteristics of an amnion tissue, comprising:
providing the amnion tissue from a human donor; and
exposing the amnion tissue to supercritical carbon dioxide to form an exposed amnion tissue, wherein the exposed amnion tissue has a higher ultimate tensile strength than the amnion tissue before exposure.

2. The method of claim 1, wherein the exposed amnion tissue has an ultimate tensile strength greater than 25 kPa.

3. The method of claim 1, wherein the exposed amnion tissue has an elastic modulus greater than 100 kPa.

4. The method of claim 1, wherein exposing the amnion tissue comprises exposing the amnion tissue to supercritical carbon dioxide at a temperature from 32° C. to 38° C.

5. The method of claim 1, wherein exposing the amnion tissue comprises exposing the soft tissue to supercritical carbon dioxide at a temperature of 35° C.

6. The method of claim 1, wherein exposing the amnion tissue comprises exposing the amnion tissue to supercritical carbon dioxide for 1.5 to 4.5 hours.

7. The method of claim 1, wherein exposing the amnion tissue comprises exposing the amnion tissue to supercritical carbon dioxide for 3 hours.

8. The method of claim 1, wherein exposing the amnion tissue comprises exposing the amnion tissue to supercritical carbon dioxide at a pressure from 1,350 to 1,475 psi.

9. The method of claim 1, wherein the method further comprises sterilizing the amnion tissue without supercritical carbon dioxide.

10. The method of claim 1, wherein the exposed amnion tissue is less likely to tear than the amnion tissue before exposure.

11. The method of claim 1, wherein exposing the amnion tissue to the supercritical carbon dioxide comprises exposing the amnion tissue to additives.

12. The method of claim 11, wherein the additives comprise peracetic acid, hydrogen peroxide, or ethanol.

13. The method of claim 11, wherein exposing the amnion tissue comprises exposing the amnion tissue to a total volume of supercritical carbon dioxide and the volume of additives is greater than or equal to 0.001% the total volume of supercritical carbon dioxide.

14. A method for treating a patient having a medical condition, comprising:
providing an amnion tissue from a human donor;
exposing the amnion tissue to a supercritical carbon dioxide to form an exposed amniotic tissue; and
administering the exposed amnion tissue to the patient.

15. The method of claim 14, wherein exposing the amnion tissue comprises exposing the amnion tissue to supercritical carbon dioxide at a temperature between 32° C. and 38° C.

16. The method of claim 14, wherein exposing the amnion tissue comprises exposing the amnion tissue to supercritical carbon dioxide at a temperature of 35° C.

17. The method of claim 14, wherein exposing the amnion tissue comprises exposing the amnion tissue to supercritical carbon dioxide for between 1.5 and 4.5 hours.

18. The method of claim 14, wherein exposing the amnion tissue comprises exposing the amnion tissue to supercritical carbon dioxide for 3 hours.

19. The method of claim 14, wherein exposing the amnion tissue comprises exposing the amnion tissue to supercritical carbon dioxide at a pressure between 1,350 and 1,475 psi.

20. The method of claim 14, wherein the method further comprises sterilizing the amnion tissue without supercritical carbon dioxide.

21. The method of claim 14, wherein the exposed amnion tissue is less likely to tear than the amnion tissue before exposure.

22. The method of claim 14, wherein exposing the amnion tissue to the supercritical carbon dioxide comprises exposing the amnion tissue to additives.

23. The method of claim 22, wherein the additives comprise peracetic acid, hydrogen peroxide, or ethanol.

24. The method of claim 22, wherein exposing the amnion tissue comprises exposing the amnion tissue to a total volume of supercritical carbon dioxide and the volume of additives is greater than or equal to 0.001% the total volume of supercritical carbon dioxide.

25. An amnion tissue composition, comprising amnion tissue, wherein:
the amnion tissue has been treated with a supercritical carbon dioxide, and
the amnion tissue composition has a higher ultimate tensile strength greater than amnion tissue not treated with the supercritical carbon dioxide.

26. The composition of claim 25, wherein the amnion tissue comprises additives.

27. The composition of claim 26, wherein the additives comprise peracetic acid, hydrogen peroxide, or ethanol.

28. The composition of claim 25, wherein the amnion tissue has an ultimate tensile strength greater than 25 kPa.

29. The composition of claim 25, wherein the amnion tissue has a higher elastic modulus than amnion tissue not treated with the supercritical carbon dioxide.

30. The method of claim 14, wherein treating the patient comprises preventing adhesions.

31. The method of claim 14, wherein administering the exposed amnion tissue to the patient comprises administering the exposed amnion tissue to a wound, a burn, a tendon, a rotator cuff, a hernia, a tissue disrupted during surgical repair, a meniscus, a nerve, a soft tissue, or a spine.

32. The method of claim 14, wherein the medical condition comprises tarsal tunnel syndrome, illiotibial band stenosis, phantom pain, a damaged meniscus, peripheral nerve damage, or peripheral nerve injury.
